(19) United States
(12) Patent Application Publication
(10) Pub. No.: US 2019/0076299 A1
(43) Pub. Date: Mar. 14, 2019

(54) METHOD FOR PRODUCING A MULTI-LAYERED FILM WEB

(71) Applicant: RKW SE, Frankenthal (Pfalz) (DE)

(72) Inventor: Ludwig Börmann, Babensham (DE)

(21) Appl. No.: 16/082,932

(22) PCT Filed: Mar. 22, 2017
(86) PCT No.: PCT/EP2017/056837

§ 371 Date: Sep. 7, 2018

(30) Foreign Application Priority Data
Mar. 22, 2016 (EP) 16161715.4

(51) Int. Cl. A61F 13/15

(52) U.S. Cl. CPC A61F 13/15577; B29C 55/06

(57) ABSTRACT

The invention relates to a process for producing a multi-layered film web from at least two starting film webs of thermoplastic polymer material, each starting film web comprising at least one low-melting polymer component and at least one high-melting polymer component. The method comprises the following steps: producing the at least two starting film webs by blown film extrusion, cast extrusion or a combination of blown film extrusion and cast extrusion; passing the at least two starting film webs up to their partly molten state, in which in each starting film web, the at least one low-melting polymer component exists in the molten liquid state, and the at least one high-melting polymer component does not exist in the molten liquid state, together over at least one heating roller, and passing the multi-layered, partly molten film web through a cooled roller nip. The invention further relates to the multi-layered film web produced with the process as well as its use.

The invention relates to a process for producing a multi-layered film web, a film web produced thereby, as well as its use, for example in the hygiene field.

In the context of environmental debates concerning conserving resources and sustainability, it is becoming of ever-increasing importance in the context of films, particularly of films for disposable products in the hygiene sector, to produce even thinner films than in the past, in order to save raw materials.

From EP-A-0 768 168 and EP-A-1 716 830, processes for the manufacture of films usable in the hygiene field are known. Having regard to their field of use, such hygiene films are required to meet several requirements. They are to be liquid-impervious and have certain haptic properties, such as softness, flexibility, low-rustling performance and textile feel. Films in the hygiene field should have a soft, cloth-like feel. In particular, when to be used for incontinence products, they should give rise to as little noise as possible, that is to say, the films should have low rustling levels. In combination with a low shininess, this results in a very textile-like film, as is desirable in the hygiene field. An additional factor is that in recent years, the absorption bodies contained in diapers and incontinence products have become progressively thinner, made possible particularly by the use of super-absorber polymers. These super-absorber polymers are employed in the form of coarsely-particulate powders, and the hygiene films must be sufficiently strong to prevent with high certainty perforation of the film by the individual particles, e.g. when subjected to loads by sitting down or other movements of the wearer. A formation of punched holes (“pinholes”) due to super-absorber polymers and a bursting of the finished film products in the packaging units must be avoided. A further requirement for hygiene films resides in a minimum tensile strength as needed for processing the film webs in the very fast-running machines (converters) of the manufacturers of e.g. diapers and sanitary napkins. This minimum tensile strength is specified in terms of 5%, 10% or 25% stretching in the machine direction (MD) or transverse direction (CD). In addition to that, films for hygiene uses should have certain strengths, for example single-layered back sheets a longitudinal tearing strength of at least 10 N/inch and a transverse tearing strength of at least 5 N/inch. If the back sheet is laminated with a non-woven, the longitudinal tearing strength should at least be 5 N/inch and the transverse tearing strength at least 2 N/inch.

The use of laminates of film and non-woven fabric is also known. A manufacture of such laminates is described in WO 2006/024394, in which a starting film web of thermoplastic polymer material is heated jointly with a starting non-woven fabric web, the melting point of which is above the crystallite melting point of the polymer material, to a temperature above the crystallite melting point of the polymer material and below the melting point of the starting non-woven fabric web, and the laminate formed is passed through a cooled roller nip and in the course thereof cooled to a temperature below the crystallite melting point of the starting film web.

In EP-A-0 768 168, a starting film web of thermoplastic polymer material is heated to a molten liquid state of the polymer material and thereafter passed through a cooled roller nip. In EP-A-1 716 830, a process including heating the polymer material and subsequent passage through a cooled roller nip is performed with a starting film web which contains a thermoplastic polymer material, including a polyethylene-matrix, in which 1 to 70 parts by weight of polypropylene, based on 100 parts by weight of polyethylene-matrix, are contained. At this, heating of the starting film web up to the molten liquid state of the polyethylene matrix material is performed, however not up to the molten liquid state of the polypropylene.

To reduce thickness of the films, stretching and drawing out film webs is known in the art. EP-A-2 565 013, for example, describes a method of stretching a starting film web of thermoplastic polymer material, which contains a low-melting polymer component and a high-melting polymer component. The method comprises heating of the starting film web up to a partly molten liquid state in which one low-melting polymer component exists in a molten liquid state and one high-melting polymer component does not exist in the molten liquid state, by means of a heating roller, and cooling down the partly molten film web by passing it through a cooled roller nip, the film web being stretched between the heating roller and the cooled roller nip.

To save raw materials, adding fillers to films is generally known. If filled films are stretched, they become breathable. To produce breathable films, films are filled with approximately 60% inert material and, after extrusion, subjected to a stretching process (usually stretching in the machine direction) in order to make the film breathable. As a filler, chalk (CaCO₃) is usually used in a particle size of 0.8-2 μm. During the stretching process, the elastic polymer portions of the film are stretched, and pores are formed at the edge of the chalk granules toward the polymer matrix. Due to the scattering of the chalk particle sizes (up to 12 μm and larger), pore sizes may also be created which can lead to leakage problems. This problem is exacerbated if for the production of breathable films that are as thin as possible, relatively high stretching levels of e.g. 2:1 to 3:1 are necessary. In some cases, films stretched in the machine direction also show low security against leakage. In addition, there is also the risk that the pores produced become too large (>1 μm) at some points in the film and thus a problem of soaking occurs (i.e. that liquid penetration resistance values (liquid impact values) greater than 3 g/m² are present). Sometimes, values for the liquid penetration resistance of less than 2 g/m² or even less than 1.5 g/m² are desirable.

Methods for producing breathable films are, for example, known from EP 0 921 943 B1, EP 1 226 013 B1, EP 1 711 330 B1 and GB 2 364 512 B. Breathable films must satisfy the above requirements with regard to mechanical properties like unfilled films and must additionally be liquid-impervious. In the context of conserving resources and sustainability, they are aimed at having low thicknesses as well.

It is known that films have a so-called memory effect. This means that films which have been stretched at, for example, 80° C. and subsequently subjected to annealing at 100° C. try to shrink when these temperatures are reached again, for example with very hot hotmelt adhesives (about 160° C.) in the converter. This problem occurs precisely in chalk-filled films because of their good thermal conductivity and in particularly thin films. If the temperature is too high or if the film thickness is too low, undesirable holes (the so-called burn-through effect) can very quickly occur.

Usually, breathable films are nowadays temporarily stored for a few days after the stretching process, and the post-crystallization is awaited before further processing such as printing is carried out because the films can shrink subsequently. Therefore, if a film is to be printed, a crystallization time of about 1 to 3 days must be waited after the stretching process and before the printing process. This process causes very high costs and hinders inline printing of the films.

Filled and stretched films tend to block on the finished rolls. Blocking means that the film layers adhere to each other due to post-shrinkage in such a way that difficulties occur during unwinding, for example that the film exhibits so-called spiral cracks. In the case of spiral cracks, the film partially adheres to the underlying film layer. This leads to tearing of the film during unwinding, which particularly affects the areas in the vicinity of the cutting mirror. Blocking is a problem especially during unwinding of thin films.

Films stretched in the machine direction (MD) have a low puncture resistance against sharp-edged super-absorber granules, which are commonly used in hygiene products for liquid absorption. Since these granules are often in direct contact with the film, pinholes and leakage may occur in the finished product. In addition, filled and MD-stretched films show low MD tear propagation strength and low MD tearing strength. The slightest damage on the roll end face or a slight blocking of the film on the roll can lead to tearing and tear propagation, resulting in spiral cracks.

The stretching process generally enhances the differences between thick and thin spots in the film and can additionally lead to edge thickening, also referred to as “neck-in”. Both effects cause so-called piston rings on the finished rolls. This means that during unwinding of these rolls, long edges or sagging are produced in the film, which may in turn lead to great difficulties in the conversion process (e.g. CD offset of the film). High degrees of stretching reinforce edge thickening (neck-in) of the film, post-shrinkage of the film after the stretching process and very low tear propagation strength of the film in the machine direction. Often, the rolls are also stored temporarily in so-called nut rolls and fed to a cutting reel only after the post-shrinkage (crystallization), in which they are then cut to the desired customer width. The post-shrinkage of the breathable films can cause a considerable layer pressure on the finished rolls, which may in turn cause blocking between the film layers and lead to spiral cracks during unwinding of the film.

Especially in back sheets (backing layers for diapers and hygienic products), edge thickening effects, such as sagging and long film edges, cause great problems when entering the converters, since on the one hand the stretching process in the machine direction strongly intensifies thick and thin spots, and, on the other hand, an offset of the foils in the transverse direction (CD) may occur, which can ultimately lead to a standstill of the converter. For this reason, it is very important that back sheets are flat when they enter converters.

Moreover, more defects or holes may occur in the films, the thinner the films become. Such holes or defects may, for example, be detected with a CCD (charge-coupled device) camera. Films with holes or defects are increasingly no longer accepted by manufacturers of finished film products and therefore constitute rejects. In addition, the continuously improving measurement technology enables easier detection of holes or defects. Therefore, there is a need for a process that reduces the number of holes or defects in thin films.

In order to solve one or several of these problems, the present invention suggests heating two starting film webs jointly up to their respective partly molten state and then to cool the obtained multi-layered film web rapidly in a cooled roller nip. By laying the two starting film webs on top of each other, the number of holes or defects in the multi-layered film web is reduced as it is highly unlikely that two defects or holes lie exactly on top of each other during heating up to the partly molten state. By the partly molten state and the subsequent cooling of the multi-layered film web, the properties of the film are significantly improved, and the above-mentioned problems are thus solved. The multi-layered film web may be stretched between the heating cylinder and the cooled roller nip. This way, the basis weight or the thickness of the film web obtained by laying two film webs on top of each other is reduced again. The basis weight or the thickness of the thicker film web may thus be compensated.

In the present case, defects or holes in a film mean defects or holes of from a diameter of 0.5 mm. Such defects or holes are, for example, visible when the film is held against the light. They may be detected with a CCD (charge-coupled device) camera or a CMOS camera system. Defects or holes are considerably larger than micropores. In the present case, the term “micropores” or “microporous” essentially refers to pores of a size of 0.1 to 5 μm. Essentially means in this regard that at least 90% of the pores, preferably 95%, more preferably 99% of the pores, or even 99.9% of the pores have a size of 0.1 to 5 μm, and the remaining pores are somewhat larger, generally up to 15 μm.

Thus, the invention relates to a process for producing a multi-layered film web from at least two starting film webs of thermoplastic polymer material, each starting film web comprising at least one low-melting polymer component and at least one high-melting polymer component, the process comprising the following steps: producing the at least two starting film webs by blown film extrusion, cast extrusion or a combination of blown film extrusion and cast extrusion; passing the at least two starting film webs up to their partly molten state jointly over at least one heating roller, wherein in each starting film web, the at least one low-melting polymer component exists in the molten liquid state, and the at least one high-melting polymer component does not exist in the molten liquid state; and passing the multi-layered, partly molten film web through a cooled roller nip.

The starting film webs may be identical or different. Two, three, four or more starting film webs may be used. Preferably, two starting film webs are used. The starting film webs may be two starting film webs produced by blown film extrusion. For example, they may be produced by producing a blown film tube, laying the tube flat, separating or slitting the tube open on the two sides where applicable, and subsequently separately or jointly feeding the two film webs to the heating roller.

In preferred embodiments of the process according to the invention, each starting film web comprises 15 to 85% by weight of low-melting polymer component and 85 to 15% by weight of high-melting polymer component, based on 100% by weight of low-melting and high-melting polymer components.

In other preferred embodiments of the process according to the invention, each starting film web comprises at least one polyethylene as the low-melting polymer component and at least one polypropylene as the high-melting polymer component.

During the process, preferably each starting film web is heated to 5 to 20° C. below the crystallite melting point of the at least one high-melting polymer component.

In exemplary embodiments of the process, the rollers forming the cooled roller nip are driven at a higher velocity than the at least one heating roller. This way, the multi-layered film web is stretched between the at least one heating roller and the cooled roller nip. Exemplary stretching ratios are at least 1:1.2, preferably at least 1:1.5, more preferably at least 1.2.

Preferably, the multi-layered film web is subjected to cooling in the cooled roller nip to at least 10 to 30° C. below the crystallite melting point of the at least one low-melting polymer component of each starting film web. Preferably, the cooled roller nip is formed by an embossing roller and a rubber roller. The film web may be printed after cooling.

In preferred embodiments, at least one starting film web contains filler. Preferably, two starting film webs contain filler. Exemplary amounts for filler are 10% to 90% by weight, preferably 20 to 80% by weight, each based on 100% by weight of the starting film web.

In exemplary embodiments, at least one starting film web is microporous. The microporous starting film web may be breathable or non-breathable.

In further exemplary embodiments, at least one starting film web is, in the course of its production, stretched in the machine or transverse direction or in the machine and transverse direction.

In addition, the invention relates to the multi-layered film webs produced with the described processes, for example with a basis weight of from 1 to 30 g/m², in particular from 5 to 25 g/m², preferably from 7 to 20 g/m², more preferably from 10 to 20 g/m², as well as their use, in particular in the hygiene or medical field, for example for back sheets in diapers, for mattress protectors or sanitary napkins. Furthermore, the invention relates to use of the produced film webs in the construction area, e.g. as cover films or as automobile protection films.

Preferred embodiments of the invention are described in the following description, the example, the figure and the claims.

In the figures,

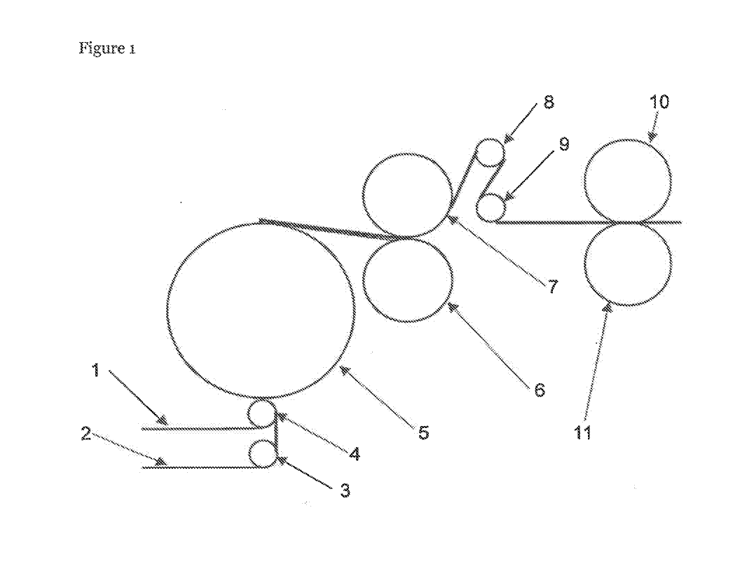

FIG. 1 shows a preferred embodiment for carrying out the process according to the invention.

In the present invention, the stated melting points, melting ranges and crystallite melting points refer to a determination according to DSC (Differential Scanning Calorimetry).

According to the invention, each starting film web contains or comprises at least one low-melting polymer component and at least one high-melting polymer component. In other words, each starting film web contains one or more low-melting polymer component(s) and one or more high-melting polymer component(s). The same meanings apply to the terms used below in the context of the invention “a low-melting polymer component” and “a high-melting polymer component”, i.e. these as well include one or more low-melting or respectively high-melting polymer component(s). Preferably, each starting film web contains one, or preferably two, low-melting polymer component(s). Preferably, it contains one, more particularly two, high-melting polymer component(s). In other embodiments of the invention, it contains preferably three low-melting polymer components and/or three high-melting polymer components. Whether a polymer material of the starting film web is to be considered a low-melting polymer component or a high-melting polymer component is determined according to the invention in terms of the respective crystallite melting point, melting point or melting range of the polymer material in relation to the heating temperature. At a given heating temperature, the liquid molten polymer materials are assigned to the low-melting polymer component and the non-liquid molten polymer materials to the high-melting polymer component.

It is well known that polymers have no sharply-defined melting point, but a melting range, even though it is possible to assign a crystallite melting point to the crystalline regions of a polymer. This crystallite melting point is always higher than the melting point or melting range of the non-crystalline components. The molten liquid state is defined by the state in which the shear modulus approaches zero. In the case of polymers having crystalline regions, the latter are then no longer detectable. The shear modulus may, for example, be determined according to ISO 6721-1 & 2. In the present invention, each starting film web is heated to a temperature at which the shear modulus of the low-melting polymer component is zero, and for the high-melting polymer component the shear modulus is not zero. No crystalline regions are then detectable any more for the low-melting polymer component and the low-melting polymer component is present in its molten liquid state. On the other hand, for the high-melting polymer component, crystalline regions are still detectable, and it is below the molten liquid state. To summarize, the shear modulus of the whole polymer material of the starting film web is accordingly not zero and crystalline regions of the high-melting polymer component are still detectable. Accordingly, there exists a partly-molten film web.

In principle, all thermoplastic polymers can be used which have the appropriate melting points to serve as materials for the two polymer components of the starting film webs. For this purpose, numerous commercial products are commercially available. Preferably, a variety of polyolefins, in particular polyethylenes, polypropylenes, copolymers of ethylene and propylene, co-polymers of ethylene and propylene with other comonomers, or mixtures thereof are employed. Furthermore, ethylene vinyl acetate (EVA), ethylene acrylate (EA), ethylene ethyl acrylate (EEA), ethylene acrylic acid (EAA), ethylene methyl acrylate (EMA), ethylene butyl acrylate (EBA), polyesters (PET), polyamides (PA), e.g. nylon, ethylene vinyl alcohols (EVOH), polystyrene (PS), polyurethane (PU), thermoplastic olefin elastomers or thermoplastic ether-ester block elastomers (TPE-E) are suitable.

The total amount of low-melting polymer component is preferably 90 to 10% by weight, in particular 90 to 20% by weight, preferably 80 to 30% by weight, more preferably 80 to 40% by weight, most preferably 70 to 50% by weight. The total amount of high-melting polymer component is preferably 10 to 90% by weight, in particular 10 to 80% by weight, preferably 20 to 70% by weight, more preferably 20 to 60% by weight, most preferably 30 to 50% by weight, each based on 100% by weight of low-melting and high-melting polymer components. In the alternative, the total amount of low-melting polymer component is preferably 85 to 15% by weight, in particular 75 to 25% by weight, and the total amount of high-melting polymer component is 15 to 85% by weight, in particular 25 to 75% by weight, again based on 100% by weight of low-melting and high-melting components. These quantitative data apply, for example, in the case of the low-melting polymer component to one or more polyethylene(s) and in the case of the high-melting polymer component to one or more polypropylene(s).

In a particularly preferred embodiment, each starting film web contains at least one polyethylene serving as the low-melting polymer component and at least one polypropylene serving as the high-melting polymer component.

Preferably, the low-melting polymer component contains ethylene polymers or consists of ethylene polymers, wherein both ethylene homopolymers as well as ethylene copolymers with ethylene as the main monomer as well as mixtures (blends) of ethylene homopolymers and ethylene co-polymers are suitable. Suitable ethylene homopolymers are LDPE (Low Density Polyethylene), LLDPE (Linear Low Density Polyethylene), MDPE (Medium Density Polyethylene) and HDPE (High Density Polyethylene). Preferred comonomers for ethylene copolymers are olefins other than ethylene with the exception of propylene, e.g. butene, hexene or octene. Preferably, in the case of the ethylene copolymers, the comonomer content is below 20% by weight, in particular below 15% by weight. In a preferred embodiment, the low-melting polymer component consists exclusively of an ethylene homopolymer or mixtures of ethylene homopolymers, e.g. of LDPE and LLDPE, which each may be contained in amounts of 10 to 90% by weight, as well as 0 to 50% by weight of MDPE. Specific examples are a polyethylene composed of 60% by weight of LDPE and 40% by weight of LLDPE or a polyethylene of 80% by weight of LDPE and 20% by weight of LLDPE.

Besides, the ethylene homopolymers and/or ethylene copolymers, the low-melting polymer component may also contain other thermoplastic polymers. There are no limits to these thermoplastic polymers as long as, as a result thereof, the temperature at which the total low-melting polymer component exists in the molten liquid state does not approach too closely the temperature at which the high-melting polymer component would be in the molten liquid state. It is also possible for the low-melting polymer component to contain a polypropylene the melting point or melting range of which is not higher than that of an ethylene homopolymer or ethylene copolymer or which, although it is higher than these, is still lower than the heating temperature to be employed. As is well-known, there exists highly-crystalline isotactic, less crystalline syndiotactic and amorphous atactic polypropylene, which have different melting points, melting ranges or crystalline melting points. When using amorphous atactic polypropylene, which has a considerably lower melting point or melting range than isotactic and, in some cases, even syndiotactic polypropylene, such might, in certain cases, as a function of the heating temperature, be assigned to the low-melting polymer component.

Preferably, the high-melting polymer component contains at least one polypropylene, the melting point, melting range or crystallite melting point of which is substantially higher than that of the low-melting polymer component. A suitable polypropylene is, in particular, isotactic polypropylene. It is also possible to employ syndiotactic polypropylene, provided that its melting point, melting range or crystallite melting point is substantially higher than that of the low-melting polymer component. Suitable polypropylenes are commercially available, for example for the manufacture of blown and/or cast films.

The high-melting polymer component may include both propylene homopolymers as well as propylene copolymers with propylene as the main monomer. In the case of propylene copolymers, the content in this context of comonomers, i.e. the non-propylene, is to be considered part of the low-melting or high-melting polymer component, depending on the other components and the heating temperature. Suitable co-monomers for propylene copolymers are olefins other than propylene, preferably ethylene. In the case of propylene-ethylene-copolymers, the ethylene content preferably is 2 to 30% by weight, particularly preferably 2 to 20% by weight and in particular 2 to 15% by weight, in which context, in practice, very good results are attained at an ethylene content of 3 to 20% by weight. These numerical values also apply to other olefins.

Below, the melting ranges for some polyethylenes and polypropylenes are listed:

LDPE: 110-114° C.;

LLDPE: 115-130° C.;

HDPE: 125-135° C.;

Propylene-homopolymers: 150-165° C.;

Propylene-ethylene-copolymers: 120-162° C., even higher temperatures being possible for very low ethylene contents;

Bimodal propylene-ethylene (homo)copolymers: 110-165° C.,

It is also possible to use so-called bimodal polypropylenes. In this context, these are two different polypropylenes, each with a different copolymer content, combined in one raw material. Such bimodal polypropylene has two crystallite melting points, in which case, as a rule, the approximate contents of the two polypropylenes can also be determined by DSC-analysis. As an example, a bimodal polypropylene is cited having crystallite melting points at 125° C. and 143° C. with a content of the two different polypropylenes of 25/75. At a heating temperature of 130° C., according to the invention, the 25% polypropylene with a crystallite melting point at 125° C. would have to be assigned to the low-melting polymer component and the 75% polypropylene having a crystallite melting point at 143° C. would have to be assigned to the high-melting polymer component.

In a particular embodiment, a starting film web is used having the following polymer components: 25 to 80% by weight, in particular 25 to 60% by weight of an LLDPE, e.g. an ethylene-octene-copolymer with 5 to 15% by weight of octene content; 20 to 30% by weight of a propylene-ethylene-copolymer with 3 to 12% by weight of ethylene; and the balance LDPE; each based on 100% by weight of low-melting and high-melting polymer components.

Just as specific molten polypropylene can be found in the low-melting polymer component, it is also possible for a specific non-molten polyethylene to be found in the high-melting polymer component, which is then assigned to the high-melting polymer component. This is illustrated by the following example. A formulation suitable for a starting film web comprises as polymer components: 30% by weight of LDPE (melting point 112° C.), 30% by weight of LLDPE (melting point 124° C.), 20% by weight of HDPE (melting point 130° C.) and 20% by weight of polypropylene (melting point 160° C.). If the film web is heated to a temperature of 126° C., the LDPE and LLDPE according to the invention are present in the molten liquid state, while not only the polypropylene, but also the HDPE are not in the molten liquid state.

The process according to the invention may also be performed with filled or microporous starting film webs.

The starting film webs for carrying out the process of the invention may be produced by any method known in the prior art. For example, the starting film web may be produced by heating the polymer components and, where applicable, fillers in an extruder, e.g. a compounding extruder, to a temperature significantly higher than the melt flow temperature of the polymer components (e.g. above 200° C.) and fusing them. This is followed by a casting method, e.g. by means of a slit nozzle, or a blow method. These methods are known in the art. In the slot nozzle method, a film is extruded through a slot nozzle. The blowing method is preferred in which a blow tube or film bubble is formed. The formed tubular film can be laid flatly on top of each other and slit open or separated at the ends so that two film webs are formed, each of which can be used as a starting film web. The advantage of slitting open or separating the tube is that air can escape. Alternatively, the flat tube may be used in the form of two starting film webs in the process of the invention without being slit or separated.

In preferred embodiments, at least one starting film web or each starting film web is stretched in the machine direction (MD), transverse direction (CD), or in the machine and transverse direction. If a microporous starting film web is used, the extruded film can be subjected to a stretching process to produce the microporosity. In addition, ring rolling is also possible.

In preferred embodiments, at least one starting film web or each starting film web is stretched. Stretching or elongating a film means stretching the film in a given direction, resulting in a reduction in the film thickness. The film can be stretched in the machine or longitudinal direction (MD), for example by a stretching unit that contains two or more rollers, e.g. three rollers, which are driven at different speeds. The film can, for example, be stretched at a stretching ratio of 1:1.5, which means that the film thickness is reduced e.g. from 15 μm to 10 μm. It is also possible, to additionally subject the film to a transverse stretching (CD). Such biaxial stretching can be achieved, for example, by stretching machines available on the market, e.g. by the company Brückner. The used stretching ratio depends on the film formulation and the chosen process parameters and can be at least 1:1.2, preferably at least 1:1.5, in particular at least 1:2, more preferably 1:2.5, even more preferably 1:3, or at least 1:4.

In preferred embodiments, at least one starting film web contains fillers. In exemplary embodiments, two starting film webs contain fillers. There are no limitations with regard to suitable fillers and they are known to the person skilled in the art. All materials are suitable which can be ground to a certain size, cannot melt in the extruder and cannot be stretched. Inorganic fillers are particularly suitable, such as chalk (calcium carbonate), clay, kaolin, calcium sulfate (gypsum) or magnesium oxide. Synthetic fillers, such as carbon fibers, cellulose derivatives, ground plastics or elastomers, are also suitable. Calcium carbonate or chalk are most preferred because of their reasonable price but also in the light of sustainability. The filler can have a particle size of e.g. 0.8 to 2 μm. If a filler of more uniform particle size than chalk is desired, it is also possible to use synthetic fillers of uniform particle size or particle size distribution. The film may also contain a small amount of fillers, e.g. 5% to 45% or 10% to 50% by weight, so that pores are formed during a stretching process, which, however, are isolated, and the film is not breathable. In order to attain breathability of the film, it is appropriate that at least 35% by weight of fillers, in particular at least 45% by weight of fillers, preferably at least 55% by weight of fillers, more preferably at least 65% by weight of fillers, based on 100% by weight of the total formulation of the starting film web including filler(s), are used. The upper limit with regard to fillers is determined in that pores are no longer formed but holes, or that the film tears off. Suitable film formulations with fillers can be determined by the person skilled in the art on a routine basis. A formulation containing 35 to 75% by weight, in particular 45 to 75% by weight of fillers, preferably 55 to 70% by weight of fillers, based on 100% by weight of starting film web, is particularly suited. Exemplary formulations for non-breathable films comprise 5 to 50% by weight, in particular 10 to 40% by weight of fillers, based on 100% by weight of starting film. web. Exemplary formulations for breathable films comprise 35 to 80% by weight, in particular from 45 to 75% by weight of fillers, based on 100% by weight of starting film web. Care must be taken in this context not to choose the content of low-melting component so high that breathability is attained but lost again because the pores close again.

If a microporous starting film web is used, it preferably has micropores in the size of from 0.1 to 5 μm, in particular of from 0.1 to 3 μm or 0.2 to 1 μm. In addition, a few larger pores can also be present.

Each starting film web may consist of one ply or a plurality of plies, it may thus be mono- and co-extruded, respectively. There is no limitation with regard to the number of plies or layers used. One or more plies or layers may be present, e.g. one ply, two plies, three plies or four plies. For example, 5, 7 or 9 plies are also possible. The plies or layers may have identical or different formulations, in which context the assignment to the low- or high-melting polymer component is in each case determined by the crystallite melting point. The plies or layers of a starting film web may be produced by co-extrusion. There is no limitation with regard to the number of the co-extruded plies or layers of a starting film web. In other embodiments, at least one starting film web or each starting film web is not co-extruded.

The starting film webs may be produced by blown film extrusion or cast extrusion or a combination thereof. For example, at least one starting film web may be produced by blown film extrusion, and at least one other starting film web by cast extrusion. There is no limitation with regard to the combination of blow-extruded and/or cast-extruded starting film webs.

In exemplary embodiments, the starting film webs may also be produced as described below:
blow-extruded;
wide-slit- or cast-extruded (mono- or co-extruded);
mono- or co-extruded;
blow-extruded, slit, on two separate webs and separate rolls, respectively;
blow-extruded, slit, on two or more separate webs at the same time;
blow-extruded, slit, laid flat as a non-slit tube;
blow-extruded, slit into two or more separate webs coming from different extruders;
cast-extruded into two or more separate webs at the same time.

There is no limitation of the number of starting film webs. There is no limitation with regard to the combination of blow-extruded or cast-extruded starting film webs. Likewise, there is no limitation with regard to the number of co-extruded layers in the combination of blow- or cast-extruded starting film webs.

It is also possible to produce the starting film webs in-line. In this case, a production step is available for the processes of extrusion and stretching (MDO, biaxial or ring rolling) as well as for the further processing (e.g. embossing and printing).

The starting film webs used in the process according to the invention may be dyed or pigmented, e.g. white with titanium dioxide. Furthermore, the starting film webs may contain conventional additives and processing aids. In particular, apart from the already mentioned fillers, this concerns pigments or other colorants, anti-adhesives, lubricants, processing aids, antistatic agents, germ-inhibiting agents (biocides), antioxidants, heat stabilizers, stabilizers with regard to UV-light or other agents for property modification. Typically, such substances, as in the case of fillers, are already added prior to the heating of the starting film web according to the invention, e.g. into the polymer melt during its manufacture or prior to extruding into a film.

The starting film webs preferably have basis weights in the range below 50 g/m², in particular below 40 g/m², preferably below 30 g/m², more preferably below 20 g/m². Basis weights in the range below 10 g/m² or below 5 g/m² are also possible. Preferred basis weights are in the range of from 1 to 30 g/m², 1 to 25 g/m² or 1 to 20 g/m², in particular of from 1 to 15 g/m², more preferably of from 2 to 10 g/m² or 7 to 20 g/m². The basis weights may also be 1 to 10 g/m², 5 to 10 g/m² or 5 to 15 g/m². The starting film webs may have thicknesses in the range of 2 to 30 μm, in particular of 2 to 15 μm, 5 to 20 μm or 5 to 10 μm.

According to the invention, the starting film webs are heated jointly by means of at least one heating cylinder and afterwards passed through a cooled roller nip. Preferably, two starting film webs are heated. The two starting film webs may be fed to the heating cylinder separately or jointly. Separated starting film webs may, for example, come from separate rolls. Joint feeding occurs, for example, if a blown tube is laid flat and not slit open or slit open in the machine direction at the two flat edges of the film, so that the flat blown tube, which represents two starting film webs, comes from one roll.

In the process according to the invention, a starting film web is fed to the heating cylinder jointly with at least one further starting film web, preferably one further starting film web. It is irrelevant which one of the starting film webs rests on the heating cylinder.

In the process according to the invention, heating of each starting film web is performed up to or above the molten liquid state of the low-melting polymer component and below the molten liquid state of the high-melting polymer component. Up to the molten liquid state means in this context that the low-melting polymer component is in a molten liquid state. It is, however, only heated to such a degree that the high-melting polymer component is not in the molten liquid state.

In order to make it possible to conduct the process in a stable manner, even for a prolonged period of time, the (crystallite) melting points of the low- and high-melting polymer components should appropriately not be too close to one another. Preferably, the crystallite melting point of the low-melting polymer component, or, in the presence of a plurality of low-melting polymer components, the crystallite melting point of those having the highest crystallite melting point, is at least about 5° C., preferably at least about 10° C. and in particular at least about 20° C. below the crystallite melting point or the molten liquid state of the high-melting polymer component or, in the presence of a plurality of high-melting polymer components, the crystallite melting point of those having the lowest crystallite melting point.

In order to attain the molten liquid state of the low-melting polymer component of the starting film webs but not the molten liquid state of the high-melting polymer component of the starting film webs, the specifically-selected difference in temperature is not subject to any specific restrictions, provided the aforesaid condition has been met. The selected temperature difference is advantageously determined by practical considerations regarding safety of the process implementation or by economic considerations. If, for example, the low-melting polymer component of each starting film web is melted at a certain temperature, further increase in temperature will not give rise to better results. Moreover, heat consumption will increase, and it is possible that one comes too close to the melting range of the high-melting polymer component of a starting film web, so that the process is more difficult to perform. Preferably, the process of the invention is performed in such a manner that heating of the starting film web is performed to 5 to 20° C., preferably 5 to 15° C. or 10 to 20° C., in particular 10 to 15° C. or 15 to 20° C., below the crystallite melting point of the high-melting polymer component of the starting film web. In the alternative, heating is performed, in particular at a temperature in the range of from 1 to 20° C., preferably 2 to 10° C., above the crystallite melting point or the molten liquid state of the low-melting polymer component(s). It must be ensured that the crystallite melting points of the low-melting polymer component(s) are attained.

According to the invention, heating of the at least two starting film webs may be performed by means of at least one heating roller. Preferably, heating is performed by means of one or more heating rollers, which may be contact rollers heated to the predetermined temperature by means of a heat carrier, such as steam, water, oil. In a preferred embodiment, a single heating or contact roller is employed. It is, however, also possible to use two or more heating rollers, in which case it is necessary to ensure that the molten liquid state of the low-melting polymer component of each starting film web is attained upstream of the cooling roller nip. In order to ensure that the starting film webs do indeed attain the temperature of the heating roller or that, in the case of high production velocities (where the surface temperature of the heating cylinder is higher than that of the films), the molten liquid state of the low-melting polymer component is attained with certainty, an adequate residence time of the starting film web on the heating roller surface must be ensured. This can be attained by an appropriate wrapping path of the heating cylinder, the diameter of the heating roller and/or the film web velocity as a function of the film thickness. It may be appropriate to use a heating roller with an anti-adhesion coated surface in order to permit easier detachment of the film web resting on the heating roller and thus prevent tearing-off of the film web. Thus, displacement of the detachment point in the direction of rotation of the heating roller is avoided and no or only a small lead is necessary. For this purpose, a PTFE (polytetrafluoroethylene) coated heating roller is used, for example.

Heating of the film webs may be supported with other heating methods, such as radiant heat, e.g. with infrared heating or infrared radiators. In addition to one or more heating rollers, a different heating, e.g. infrared heating, may be provided.

According to the invention, the multi-layered film web is passed through a cooled roller nip after heating. The rollers forming the cooling roller nip are cooled in such a manner that rapid and sudden cooling is attained. Cooling to a temperature below the crystallite melting point of the low-melting polymer component of at least one starting film web, preferably of each starting film web, preferably to at least 5° C. below that melting point, in particular to at least 10° C. below that melting point, is appropriate. Preferred cooling ranges are 5 to 10° C., more preferably 10 to 30° C. below the crystallite melting point of the low-melting polymer component of one starting film web or each starting film web. Cooling of the rollers with water may, for example, take place in a temperature range of 5 to 20° C., e.g. using water having a temperature of about 10° C. The spacing between the heating roller or, if a plurality of heating rollers are used, the last heating roller and/or other heating sources and the cooling roller nip is not too wide in this context, due to possible heat loss.

The cooling roller nip may in the simplest case be, for example, a smooth-roller nip with two smooth rollers. In the case of hygiene films, the roller nip is preferably formed by a pair of rollers with one texturing roller and one smooth roller (i.e. a rubber roller), thereby imparting to the film web a textured surface. Preferred textures in the hygiene field are micro-textures, e.g. a truncated pyramid. Preferably, the cooled roller nip consists of a steel roller and a rubber roller operating under counter-pressure, the steel roller being provided with the textured surface. The steel roller may be provided with a textile-like engraving which reinforces the textile appearance of the surface of the film. An embossed structure of the steel roller further reduces the shininess of the film.

The velocity of the rollers forming the cooling roller nip may be selected such that said velocity is the same as that of the heating roller or, if a plurality of heating rollers are used, the same as that of the last heating roller, so that the film is not stretched between them. The velocity of the rollers forming the cooling roller nip may also be selected such that said velocity is higher or lower than that of the heating roller or, if a plurality of heating rollers are used, higher or lower than that of the last heating roller, so that the film is stretched or shrunk between them. Due to heat loss, the spacing between the heating roller and the cooling roller nip should be kept as small as possible.

In preferred embodiments, the multi-layered film web is stretched between the heating cylinder and the cooling roller nip. It is essential that the film web is in the partly molten state during this stretching procedure. The stretching ratio depends on the film formulation and the selected process parameters and is preferably at least 1:1.2, more preferably at least 1:1.5, in particular at least 1:2, even more preferably at least 1:2.5, more preferably at least 1:3, or at least 1:4.

In a preferred embodiment of the invention, the stretching is brought about in that the cooling rollers forming the cooled roller nip are driven at a higher velocity than the heating roller. In another preferred embodiment of the invention, two or more rollers, of which at least two are driven at different velocities, are provided upstream of the cooling roller nip such that the film web is stretched between these two rollers, and in which case at least the first of the two or more rollers is designed as a heating roller. It is also possible for the second and, where applicable, the further rollers to be likewise designed as a heating roller. In particular, if a plurality of rollers are provided, it is, however, also possible for one of the rollers to be designed as a cooling roller. A cooling roller brings about cooling of the film web on one side and, therefore, results in slow cooling of the film. In contrast thereto, the cooling roller nip provided according to the invention, due to the two cooling rollers, provides cooling of the film web on both sides, thereby causing fast cooling. If one cooling roller is employed, heating to the partially-molten state of the film web upstream of the cooling roller nip is again necessary, which can appropriately again be performed by a heating roller. Thus, arrangements such as heating roller—heating roller—cooled roller nip or heating roller—cooling roller—heating roller—cooled roller nip are possible.

Depending on the film parameters and other process conditions, the film web velocities are in the range of 50 to 900 m/min. The velocity of the heating roller(s) is preferably 50 to 900 m/min, in particular 50 to 800 m/min, preferably 100 to 600 m/min. The velocity of the rollers forming the cooling roller nip is preferably 50 to 900 m/min, in particular 50 to 800 m/min, preferably 100 to 600 m/min. The velocities of the heating roller(s) and the cooling rollers are selected such that, depending on the film formulation and the selected process parameters, said velocities are the same or, however, different, so that the film is stretched or shrunk (annealing) in the desired ratio.

The process according to the invention enables the manufacture of multi-layered films having very small basis weights of e.g. 2, 3, 4, 5, 6, 7, 8, 9, 10, 11, 12, 13, 14, 15, 16, 17, 18, 19, 20 or 25 g/m². The corresponding film thicknesses lie within the range of e.g. 25, 20, 19, 18, 17, 16, 15, 14, 13, 12, 11, 10, 9, 8, 7, 6 or only 5 μm. Preferred films have a thickness in the range of from 2 to 13 μm or 4 to 25 μm or have a basis weight of from 1 to 15 g/m² or of from 4 to 25 g/m² or of from 7 to 20 g/m².

Despite being very thin and microporous, the films obtained according to the invention have excellent mechanical properties and, in addition, still have a very high puncture resistance (i.e. resistance to super-absorber granules, e.g. in diapers) and high thereto-stabilities (i.e. resistance to hot melt-adhesives).

Multi-layered films obtained according to the invention may be further processed in a known manner. For example, single back sheets or non-woven fabric-film laminates can be produced therefrom. For manufacturing non-woven fabric-film laminates, the films may be adhesively-bonded to non-wovens by adhesive agents, preferably in-line. Apart from that, non-woven fabric-film laminates may also be manufactured by thereto-bonding, known to the person skilled in the art, in which case the material of a film and/or non-woven fabric obtained according to the invention is melted by high temperature and pressure at particular points by two heated rollers, in most cases an embossing roller (engraved steel roller) and a smooth steel roller serving as counter-roller, thereby causing the film and non-woven fabric to be bonded together. Moreover, non-woven fabric-film laminates, as described above, may also be manufactured by thermo-laminating. Thermo-laminating is particularly preferred in the case of very thin films, e.g. under 10 g/m² or e.g. 4 g/m². In addition, non-woven fabric-film laminates may also be produced by means of ultrasonic lamination (e.g. using ultrasound Herrmann technology). The non-woven fabric-film laminates produced may be further processed in a manner known per se, in which case stretching in the machine or transverse direction or in both directions is likewise possible. Single back sheets may also be processed further.

FIG. 1 shows a preferred embodiment for carrying out the process according to the invention. A starting film web 2 is passed over a deflecting roller 3, and a starting film web 1 is passed over a deflecting and pressing roller 4 onto a heating cylinder 5. The heating cylinder 5 or the heating roller 5 is, for example, an anti-adhesively coated steel roller, which is heated to the desired surface temperature by heat supply. There, according to the invention, both webs are heated to the partly molten state and conjoin into a multi-layered film web. The film web runs from the heating roller 5 into a cooling roller nip formed by the rollers 6 and 7. The roller 6 is preferably designed as a structure or embossing roller, thereby imparting an embossed structure or structured surface to the film web. The roller 7 is preferably a rubber roller. The roller pair 6/7 is preferably water-cooled, e.g. using water having a temperature of about 10° C. The rollers 6 and 7 forming the cooling nip are driven such that a higher, lower or the same velocity arises in relation to the web velocity of the heating roller 5. In the cooling roller nip, the film web is abruptly cooled and embossed. Downstream of the roller pair 6/7, the film can be directly taken off, or, via the deflecting rollers 8 and 9, which may also be cooled, the film web may, for example, be subjected to stretching by means of the ring rolling rollers 10 and 11. The finished film web may be further processed in a manner known per se.

Due to the manufacture of films with thin thicknesses, the invention enables raw material savings, thereby contributing to saving resources and sustainability. As a result, it contributes to protecting the environment. This applies to films in the hygiene sector and to other applications, especially applications where the films are used to a large extent as components of disposable products.

In view of the problems in the prior art described above, the film manufactured according to the invention offers the following improvements and advantages:
The film allows a high temperature load, e.g. with hot melt adhesives.
The film shows almost no post-shrinkage.
Since the films show hardly measurable post-shrinkage, they can be imprinted inline directly after the stretching process, for example by means of an intermediate “hot embossing process”.
At high thermal loads, for example when hot melt adhesive systems are applied, the film shows higher resistance and low shrinking behavior, respectively, and smaller so-called burn-through effects, respectively; this way, the formation of holes is reduced or does not occur any longer.

It is also possible to perform the process described herein in such a way that of the at least two starting film webs, only one starting film web is in the partly molten state.

The films obtained according to the invention can be used in many areas. They are used in the hygiene or medical field, e.g. as an underwear protection film or generally as a liquid-impermeable barrier layer, in particular as back sheets in diapers, sanitary napkins, mattress protectors or similar products. Furthermore, the films can be used in other technical fields, for example in the construction sector as construction films, e.g. for roof lining webs, screed coverings or wall coverings, or as car protection films in the automotive area.

Films obtained according to the invention may he further processed in a known manner, for example into non-woven fabric-film laminates. For manufacturing such laminates, the latter may be adhesively-bonded by adhesive agents, preferably in-line. Apart from that, non-woven fabric-film laminates may also be manufactured by thermo-bonding, known to the person skilled in the art, in which case the material of a film and/or non-woven fabric obtained according to the invention is melted by high temperature and pressure at particular points by two heated rollers, in most cases an embossing roller (engraved steel roller) and a smooth steel roller serving as counter-roller, thereby causing the film and non-woven fabric to be bonded together. Moreover, non-woven fabric-film laminates may also be manufactured by thermo-laminating, for example as described in EP 1 784 306 B1. Thermo-laminating is particularly preferred in the case of very thin films, e.g. under 4 g/m². Alternatively, non-woven fabric-film laminates may also be produced by means of ultrasonic lamination (e.g. using ultrasound Herrmann technology). The manufactured non-woven fabric-film laminates may be further processed in a manner known per se.

By laying the starting film webs on top of each other on the heating cylinder, the process according to the invention enables a large reduction of the number of defects and holes. At the same time, the starting film webs are joined together and thermally treated by means of the partly molten state. Stretching the multi-layered film web, for example between the heating cylinder and the cooling roller nip, results in reduction of the basis weight or the thickness of the film web, so that the potential disadvantage of a higher basis weight caused by using at least two starting film webs may be compensated. All in all, the invention reduces rejects of thin polymer films and thus makes an important contribution to saving resources and sustainability.

The invention is explained in detail by way of the following example, without limiting the invention.

### EXAMPLE

The starting film webs are produced by common blown film extrusion at an extruder temperature of 240° C. using a formulation according to Table I.

TABLE I

| Amount in | | | Crystallite |
|---|---|---|---|
| parts by | | Density, | melting point |
| weight | Component | g/cm³ | ° C. |
| 59 | LDPE | 0.922-0.924 | 113 |
| 41 | LLDPE octene | 0.930 | 124 |
| 30 | Polypropylene¹⁾ | 0.90 | 162 |
| 5 | TiO₂-white- | 1.69 | — |
| | concentrate | | |

¹⁾Propylene-ethylene-copolymer with 10% by weight of ethylene
² 190° C./2.16 kg for LDPE and LLDPE and 230° C./2.16 kg for polypropylene

The blown tube with a basis weight of 10.4 g/m² (corresponding to a film thickness of 11 μm) was laid flat and slit open at the two sides, resulting in two film webs. According to the process according to the invention, the two starting film webs were fed to a heating cylinder, as shown in FIG. 1 with the starting film webs 1 and 2. The surface temperature of the heating cylinder was 130° C. This way, both starting film webs were heated such that each of them was in the partly molten state. Subsequently, the obtained two-layered film web was fed to a cooled roller nip (water-cooled with 10-15° C.). The rollers of the cooling roller nip were driven at a higher web velocity than the heating roller, so that the film web was stretched. The stretching level results from the differential speed between the heating roller and the cooling roller nip. The two-layered film web was stretched at three different stretching levels, with the following basis weights having been obtained for the film web:
77% stretching level; stretching ratio 1:1.43; basis weight 14.5 g/m² (20.8:1.43);
83% stretching level; stretching ratio 1:1.72; basis weight 12.1 g/m² (20.8:1.72);
136% stretching level; stretching ratio 1:2.08; basis weight 10.0 g/m² (20.8:2.08).

When measuring the film web with a CCD camera, the camera indicated that the two-layered film web had 95% less holes than the single-layered blown starting web. In addition, the properties of the film were the same or better than those of prior art films (for example tensile strength, tear strength, elongation at break, puncture resistance).

1. A process for producing a multi-layered film web from at least two starting film webs of thermoplastic polymer material, each starting film web comprising at least one low-melting polymer component and at least one high-melting polymer component, the process comprising the following steps:
producing the at least two starting film webs by blown film extrusion, cast extrusion or a combination of blown film extrusion and cast extrusion,
passing the at least two starting film webs up to their partly molten state, in which in each starting film web, the at least one low-melting polymer component exists in the molten liquid state, and the at least one high-melting polymer component does not exist in the molten liquid state, jointly over at least one heating roller, and
passing the multi-layered, partly molten film web through a cooled roller nip.

2. The process according to claim 1, wherein the at least two starting film webs are identical or different.

3. The process according to claim 1, wherein the at least two starting film webs are two starting film webs produced by blown film extrusion, wherein a blown film tube is produced, the tube is laid flat, separated at the two sides if applicable, and wherein the two film webs are fed to the heating roller separately or jointly.

4. The process according to claim 1, wherein each starting film web comprises 15 to 85% by weight of low-melting polymer component and 85 to 15% by weight of high-melting polymer component, based on 100% by weight of low-melting and high-melting polymer components.

5. The process according to claim 1, wherein each starting film web comprises at least one polyethylene as the low-melting polymer component and at least one polypropylene as the high-melting polymer component.

6. The process according to claim 1, wherein each starting film web is heated to 5 to 20° C. below the crystallite melting point of the at least one high-melting polymer component.

7. The process according to claim 1, wherein the rollers forming the cooled roller nip are driven at a higher velocity than the at least one heating roller.

8. The process according to claim 1, wherein the multi-layered film web is stretched between the at least one heating roller and the cooled roller nip, at a stretching ratio of at least 1:1.2.

9. The process according to claim 1, wherein the multi-layered film web is subjected to cooling in the cooled roller nip to at least 10 to 30° C. below the crystallite melting point of the at least one low-melting polymer component of each starting film web.

10. The process according to claim 1, wherein the starting film webs contain filler, in an amount of 10% to 90% by weight, each based on 100% by weight of the starting film web.

11. The process according to claim 10, wherein at least one starting film web is microporous.

12. The process according to claim 1, wherein at least one starting film web is, in the course of its production, stretched in the machine or transverse direction or in the machine and transverse direction.

13. Multi-layered film web, obtainable by a process according to claim 1.

14. The multi-layered film web according to claim 13, having a basis weight within the range of from 1 to 30 g/m².

15. Use of the multi-layered film web according to claim 13 in the hygiene or medical field.

16. The process according to claim 1, wherein the multi-layered film web is stretched between the at least one heating roller and the cooled roller nip, at a stretching ratio of at least 1:1.5.

17. The process according to claim 1, wherein the multi-layered film web is stretched between the at least one heating roller and the cooled roller nip, at a stretching ratio of at least 1.2.

18. The process according to claim 1, wherein the starting film webs contain filler, in an amount of 20% to 80% by weight, each based on 100% by weight of the starting film web.

19. The multi-layered film web according to claim 13, having a basis weight within the range of from 5 to 25 g/m².

20. The multi-layered film web according to claim 13, having a basis weight within the range of from 7 to 20 g/m².

21. The multi-layered film web according to claim 13, having a basis weight within the range of from 10 to 20 g/m².
